(19) United States
(12) Patent Application Publication
(10) Pub. No.: US 2013/0112194 A1
(43) Pub. Date: May 9, 2013

(54) PROCESS FOR PROVIDING A FILLED CANISTER FOR AN INHALER

(71) Applicants: Simon Christopher Berry, Molndal (SE); Agnes Claude Marcelle Pierrette Colombani, Molndal (SE); Ian Mark Fletcher, Molndal (SE); Duncan Charles Dickie, Macclesfield (GB); Andrew John Ludzik, Macclesfield (GB); Darren James Hodson, Macclesfield (GB); Robert Bernard Jansen, Macclesfield (GB); Katherine Jane Lee, Macclesfield (GB); Julie Page, Macclesfield (GB)

(72) Inventors: Simon Christopher Berry, Molndal (SE); Agnes Claude Marcelle Pierrette Colombani, Molndal (SE); Ian Mark Fletcher, Molndal (SE); Duncan Charles Dickie, Macclesfield (GB); Andrew John Ludzik, Macclesfield (GB); Darren James Hodson, Macclesfield (GB); Robert Bernard Jansen, Macclesfield (GB); Katherine Jane Lee, Macclesfield (GB); Tim Page, Macclesfield (GB)

(21) Appl. No.: 13/577,766

(22) PCT Filed: Feb. 9, 2011
(86) PCT No.: PCT/GB11/50222

§ 371 Date: Jan. 23, 2013

Related U.S. Application Data
(60) Provisional application No. 61/302,988, filed on Feb. 10, 2010.

(51) Int. Cl. B65B 31/00; A61M 16/12; A61M 15/00; A61M 11/04

(52) U.S. Cl. CPC B65B 31/003; A61M 11/04; A61M 16/12; A61M 15/0091
USPC 128/200.23; 141/3; 128/203.12

(57) ABSTRACT

A process for filling a canister for an inhaler is provided. The canister may be filled with components of a medicament. The canister may be suitable for use in a pressurised metered dose inhaler, such as a breath-actuate inhaler. The process may include sealing the canister with air at ambient conditions. A filling device may dispense a pressurised liquid or gas into the sealed, air-filled canister through a metering valve. The pressurised liquid or gas may comprise at least a propellant. While pressure-filled canisters were previously purged with propellant immediately before sealing to remove the air, the present process may exclude the purging step.

The present invention relates to a process for providing a filled canister for an inhaler, in particular a process for supplying a canister and filling the canister with components of a medicament, which canister is suitable for use in a pressurised metered dose inhaler.

Inhalers, such as dry powder inhalers (DPIs) and pressurised metered dose inhalers (pMDIs), are commonly used for delivery of a wide range of medicaments. A pMDI comprises at least one canister of medicament, the canister being actuated, e.g., by opening a metering valve, to deliver a dose of medicament through a mouthpiece to a user. The inhaler may be actuated manually and/or may be provided with an actuation mechanism to actuate the canister automatically, e.g. a breath-actuated mechanism that operates in response to inhalation by a user. Such breath-actuated inhalers (BAIs) ensure that a dose of medicament dispensed on actuation of the canister is supplied whilst the user is inhaling, which is particularly useful for those who may find it difficult to co-ordinate the dispensing of a dose of medicament with inhaling the dose.

A typical medicament for a pMDI comprises at least one active pharmaceutical ingredient (API) and preferably any one or more of a propellant (preferably one of the more ozone-friendly propellants approved for inhalation such as 1,1,1,2-tetrafluoroethane (HFA 134a) or 1,1,1,2,3,3,3-heptafluoropropane (HFA 227)) and any other suitable component(s), such as surfactant(s), co-solvent(s), lubricant(s), etc. The medicament may be a suspension or a solution, or a mixture thereof.

A canister with a medicament, suitable for use in a pMDI, can be provided by one of a number of conventional processes. Typically, canisters are provided to a filling line or stage, to be filled with a suitable medicament. In conventional systems, a canister may undergo one or more appropriate preparation steps prior to the filling stage, such as cleaning by blowing with compressed air and/or vacuum suction, and purging with propellant, etc. The canister is then filled with an appropriate and precise amount of medicament, which is typically metered into the canister by weight or volume. The canister may instead or additionally be weighed after filling to ensure an accurate amount of medicament is present in the canister.

There are several known processes for filling a pMDI canister with medicament in solution or suspension, including cold filling and pressure filling (typically single-stage or two-stage). In a cold filling process, volatile propellant is cooled to below its boiling point and is thus liquefied. The required API(s) is typically mixed with the liquefied propellant, along with any other components of the medicament, and the medicament is provided to a filling nozzle from which it is dispensed into a canister in a filling line. The canister is then sealed, typically with any suitable metering valve. The disadvantage of this process is the need to precisely control and maintain the low temperatures of all the stages of the system (particularly the mixing vessel and the pipes therefrom to the nozzle, etc.). Such cold temperature systems are costly and difficult to operate and introduce problems with condensation of water vapour in the medicament. Furthermore any fluctuations in temperature may affect the concentration of the dose provided by the canister in use, which is seriously detrimental for a medicament.

Pressure filling is an alternative process for supplying a medicament to a canister. This process is advantageous compared with cold filling as it does not require the system components to be cooled to temperatures low enough to liquefy a propellant.

In both the pressure filling and cold filling methods it has long been thought that a purging step, as discussed above, is necessary to firstly ensure that the pressure in the filled canister is not too high (as it was thought that it might lead to canister bursting during or after filling), also to ensure that oxygen is substantially removed from the canister to prevent any chemical degradation of the product due to oxidation, and further to ensure that moisture is substantially removed from the canister.

Cold filling a canister has the perceived advantage that a volatile liquid self-purges the canister of air (because some of the propellant will inevitably boil off and expel air from the canister before the valve is crimped on), whereas typically the pressure filling process does not self-purge (unless, in the two-stage process, the concentrate contains a volatile, heavier than air component).

Therefore the standard pressure filling process (whether single-stage or two-stage) includes purging as a first process step, immediately prior to canister filling. The purging step comprises adding typically a few drops of liquefied propellant to the empty canister, which rapidly boils (on contact with the warm canister) and forces air out of the canister, which is then ready to be pressure filled immediately afterwards as disclosed above. This is a disadvantage, because introducing any additional step is time consuming and more costly and furthermore requires release of excess propellant into the surroundings.

In a pressure filling process, the propellant is liquefied under pressure. In a common two-stage pressure filling process, the API(s) and typically any other components of the medicament (e.g. co-solvent(s), surfactant(s), non-volatile liquid(s), etc.) are pre-mixed into a concentrate that is filled into an empty canister. The concentrate may be cooled if required. The canister is then sealed with a metering valve and the liquefied propellant is injected into the sealed canister via the valve, mixing with the concentrate to produce the desired medicament. A typical single-stage pressure filling process is similar to a two-stage process, except that the concentrate is also pre-mixed with the propellant under pressure, and the mixture is injected into a sealed canister via the metering valve.

More details of known canister filling processes are disclosed in the art, e.g. in Metered Dose Inhaler Technology pp 79-107 (Tol S. Purewal & David J. W. Grant, eds., 1998).

According to the present invention, there is provided a process for providing a filled canister containing a medicament for an inhaler which overcomes the drawbacks of the prior art. This is achieved by the process as defined in the independent claims.

From a first broad aspect, there is provided a process for providing a filled canister for an inhaler, the process comprising the steps of:
providing a canister, the canister comprising an enclosure suitable for containing a medicament and having an opening for receiving the medicament, and the canister being substantially filled with air at ambient conditions;
sealing the opening of the air-filled canister by securing thereto a metering valve or other sealing means;
providing the sealed, air-filled canister to a filling device; and
dispensing from the filling device a pressurised liquid and/or gas into the sealed, air-filled canister through the metering valve or other sealing means, the pressurised liquid and/or gas comprising at least a propellant,

wherein:
the sealed canister is substantially devoid of propellant prior to the step of dispensing the pressurised liquid and/or gas; and
the sealed canister is substantially filled with at least a first proportion of propellant and a second proportion of air after the step of dispensing the pressurised liquid and/or gas.

The present invention further extends to a canister filled with at least some components of a medicament according to the process of the present invention, and to an inhaler comprising a canister filled with components of a medicament according to the process of the present invention.

Embodiments of the present invention are defined in the dependent claims.

The present invention is advantageous because the significant cooling requirements of a cold filling process are not required, yet the purging step of a pressure filling process, and its associated disadvantages, are also avoided. Furthermore a problem with conventional pMDI devices is that a reduction in actuation weight of a subsequent dispensed aerosol may occur if the metering valve of the device is held in an actuated or open position for an extended period of time after the previous actuation. It has surprisingly been found that this effect is significantly reduced if the canister is unpurged before it is filled with medicament. Thus, according to embodiments of the present invention, the canister is not purged, as it would be conventionally. Rather the canister remains filled with the ambient gas, i.e. air, and is sealed with a metering valve (in an airtight manner when the valve is in its closed position) whilst still filled with air. Namely no deliberate or significant amount of propellant, or other purging fluid or gas, is intentionally introduced into the canister at any stage (to remove the air) prior to sealing the canister, and the canister is filled with propellant (or propellant mixed with components of a medicament) whilst the canister still contains air. Such a canister, that has undergone no deliberate purging process (to remove or expel air) prior to being sealed and filled, is termed an unpurged canister. In some embodiments, an active pharmaceutical ingredient (API) and/or other components of a medicament may be added prior to sealing the canister with a metering valve, but for the avoidance of doubt this does not include any propellant for the purposes of purging as the canister is unpurged.

The applicant has surprisingly determined that the pressure in an unpurged canister (i.e. a canister that has not been purged with volatile propellant prior to filling with medicament as in a conventional pressure filling process) does not exceed safe limits, contrary to the teachings of the prior art. It has furthermore been surprisingly determined that the presence of oxygen is not detrimental for many products. Still further, the applicant has determined that the amount of water typically trapped in a canister may be reduced by controlling the local environment around the filling machine. Thus, for many products, the applicant has unexpectedly determined that purging is an unnecessary step. Removal of the purging process step advantageously reduces the quantity of, e.g. HFA, propellant released into the atmosphere as a result of the filling process (to ensure complete purging it is standard practice to add a small overage of propellant to the canister, and a small quantity of the propellant may be released to the atmosphere for every canister). Furthermore, removing the purging step increases the efficiency and reliability of the filling process due to the elimination of one of the process steps.

As discussed above, the applicant has advantageously determined that unpurged canisters are suitable for use in pMDIs, particularly breath-actuated inhalers, and that undesirable release of propellant in the can supply and filling process is thereby minimised. Furthermore, the undesirable reduction in actuation weight that may occur in a subsequent actuation, when a metering valve has remained open for an extended period of time, is minimised. This is particularly advantageous for devices where the metering valve can be held in an open condition, such as manually operated devices which may be held in the actuated or open position by the patient, or those that have, e.g., a catch and release mechanism after firing, or automatically operated devices such as a breath-actuated inhaler where the actuation force is reset, in some cases manually by the patient, after firing. Without intending to be bound by theory, it is postulated that this advantage may be achieved by the higher pressure in unpurged canisters, relative to conventional purged canisters, resulting in better filling of the metering valve chamber even after the valve has been held in the actuated position for an extended period of time.

The applicant has further determined an alternative process for providing a canister containing a medicament for an inhaler, in particular a process for supplying a canister and filling the canister with a medicament (or components thereof), suitable for a pressurised metered dose inhaler. The alternative process also minimises the undesirable release of propellant in the can supply and filling process, but not by providing sealed canisters that are substantially devoid of propellant (i.e. unpurged canisters). Rather, and in accordance with a further broad aspect of the present invention, there is provided a novel process for purging and filling a canister, the canister for use in an inhaler, the process comprising the steps of:
providing a canister, the canister comprising an enclosure suitable for containing a medicament and having an opening for receiving the medicament, and the canister being substantially filled with air;
dispensing an amount of a substance, the substance being any substance excluding a propellant and preferably being an inert substance, into the canister so as to displace a substantial proportion of the air thereby providing a canister substantially filled with the substance;
sealing the opening of the canister by securing thereto a metering valve or other sealing means, sealing the substance therein;
providing the sealed canister to a filling device; and
dispensing from the filling device a pressurised liquid and/or gas into the sealed canister through the metering valve or other sealing means, wherein:
the pressurised liquid and/or gas comprises at least a propellant, thereby providing a sealed canister containing at least first proportion of propellant and a second proportion of the substance.

The substance may be any suitable substance, except for a propellant. Preferably the substance is any substance excluding an HFA propellant or a CFC propellant, more preferably excluding HFA 227 or HFA 134a. Preferably the substance comprises an inert substance such as nitrogen or argon or may comprise carbon dioxide. The substance is preferably in gaseous and/or liquid form. The substance may be at ambient pressure or the substance may be pressurised. The substance may be at ambient temperature or may be cooled.

The canister of any of the above aspects may be any suitable canister for storing a medicament. Preferably the canister comprises a material such as aluminium, glass or the like. Preferably the canister is coated, preferably at least a portion of the internal surface and more preferably substantially the entire internal surface of the canister is coated. The coating may comprise any material or composition that is suitable for use in contact with a medicament. In a preferred embodiment, the coating comprises a polymer or a polymer blend. Preferably the coating comprises a fluoropolymer. The coating preferably comprises perfluoroalkoxyethylene (PFA), polytetrafluoroethylene (PTFE), fluorinated ethylene propylene (FEP), PET or the like. The coating may be applied by any suitable technique. Preferably the can coating is applied by any suitable method such as dipping, dry powder coating, spraying or preferably plasma coating. The canister may be pre-heated before the coating is applied and/or may be heated after the coating is applied to sinter or anneal the coating.

The medicament may contain various active ingredients. The active ingredient may be selected from any therapeutic or diagnostic agent. For example, the active ingredient may be an antiallergic, a bronchodilator (e.g. a beta2-adrenoceptor agonist or a muscarinic antagonist or a single compound having both these properties), a bronchoconstrictor, a pulmonary lung surfactant, an analgesic, an antibiotic, a mast cell inhibitor, an antihistamine, an anti-inflammatory, an antineoplastic, an anaesthetic, an anti-tubercular, an imaging agent, a cardiovascular agent, an enzyme, a steroid, genetic material, a viral vector, an antisense agent, a protein (such as insulin), a peptide, a non-steroidal glucocorticoid Receptor (GR Receptor) agonist, an antioxidant, a chemokine antagonist (e.g. a CCR1 antagonist), a corticosteroid, a CRTh2 antagonist, a DP1 antagonist, an Histone Deacetylase Inducer, an IKK2 inhibitor, a COX inhibitor, a lipoxygenase inhibitor, a leukotriene receptor antagonist, an MPO inhibitor, a p38 inhibitor, a PDE inhibitor, a PPARγ agonist, a protease inhibitor, a statin, a thromboxane antagonist, a vasodilator, an ENAC blocker (Epithelial Sodium-channel blocker) and combinations thereof.

Examples of specific active ingredients that can be incorporated in the medicament include:
(i) antioxidants:—Allopurinol, Erdosteine, Mannitol, N-acetyl cysteine choline ester, N-acetyl cysteine ethyl ester, N-Acetylcysteine, N-Acetylcysteine amide and Niacin;
(ii) chemokine antagonists:—BX471 ((2R)-1-[[2-[(aminocarbonyl)amino]-4-chlorophenoxy]acetyl]-4-[(4-fluorophenyl)methyl]-2-methylpiperazine monohydrochloride), CCX634, N-{2-[((2S)-3-{[1-(4-chlorobenzyl)piperidin-4-yl]amino}-2-hydroxy-2-methylpropyl)oxy]-4-hydroxyphenyl}acetamide (see WO 2003/051839), and 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid (see WO 2008/010765), 656933 (N-(2-bromophenyl)-N′-(4-cyano-1H-1,2,3-benzotriazol-7-yl)urea), 766994 (4-({[({[(2R)-4-(3,4-dichlorobenzyl)morpholin-2-yl]methyl}amino)carbonyl]-amino}methyl)benzamide), CCX-282, CCX-915, Cyanovirin N, E-921, INCB-003284, NCB-9471, Maraviroc, MLN-3701, MLN-3897, T-487 (N-{1-[3-(4-ethoxyphenyl)-4-oxo-3,4-dihydropyrido[2,3-d]pyrimidin-2-yl]ethyl}-N-(pyridin-3-ylmethyl)-2-[4-(trifluoromethoxy)phenyl]acetamide) and Vicriviroc
(iii) Corticosteroids:—Alclometasone dipropionate, Amelometasone, Beclomethasone dipropionate, Budesonide, Butixocort propionate, Ciclesonide, Clobetasol propionate, Desisobutyrylciclesonide, Etiprednol dicloacetate, Fluocinolone acetonide, Fluticasone Furoate, Fluticasone propionate, Loteprednol etabonate (topical) and Mometasone furoate.
(iv) DP1 antagonisits:—L888839 and MK0525;
(v) Histone deacetylase inducers:—ADC4022, Aminophylline, a Methylxanthine or Theophylline;
(vi) IKK2 inhibitors:—2-{[2-(2-Methylamino-pyrimidin-4-yl)-1H-indole-5-carbonyl]-amino}-3-(phenyl-pyridin-2-yl-amino)-propionic acid;
(vii) COX inhibitors:—Celecoxib, Diclofenac sodium, Etodolac, Ibuprofen, Indomethacin, Meloxicam, Nimesulide, OC1768, OC2125, OC2184, OC499, OCD9101, Parecoxib sodium, Piceatannol, Piroxicam, Rofecoxib and Valdecoxib;
(viii) Lipoxygenase inhibitors:—Ajulemic acid, Darbufelone, Darbufelone mesilate, Dexibuprofen lysine (monohydrate), Etalocib sodium, Licofelone, Linazolast, Lonapalene, Masoprocol, MN-001, Tepoxalin, UCB-35440, Veliflapon, ZD-2138, ZD-4007 and Zileuton ((±)-1-(1-Benzo[b]thien-2-ylethyl)-1-hydroxyurea);
(ix) Leukotriene receptor antagonists:—Ablukast, Iralukast (CGP 45715A), Montelukast, Montelukast sodium, Ontazolast, Pranlukast, Pranlukast hydrate (mono Na salt), Verlukast (MK-679) and Zafirlukast;
(x) MPO Inhibitors:—Hydroxamic acid derivative (N-(4-chloro-2-methyl-phenyl)-4-phenyl-4-[[(4-propan-2-ylphenyl)sulfonylamino]methyl]piperidine-1-carboxamide), Piceatannol and Resveratrol;
(xi) Beta2-adrenoceptor agonists:—metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol, indacaterol, salmeterol (e.g. as xinafoate), bambuterol (e.g. as hydrochloride), carmoterol, indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide; 3-(4-{[6-({(2R)-2-hydroxy-2-[4-hydroxy-3-(hydroxy-methyl)phenyl]ethyl}amino)-hexyl]oxy}butyl)benzenesulfonamide; GSK 159797, GSK 159802, GSK 597901, GSK 642444, GSK 678007; and a compound selected from N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide, N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one, and N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. wherein the counter ion is hydrochloride (for example a monohydrochloride or a dihydrochloride), hydrobromide (for example a monohydrobromide or a dihydrobromide), fumarate, methanesulphonate, ethanesulphonate, benzenesulphonate, 2,5-dichlorobenzenesulphonate, p-toluenesulphonate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), D-mandelate, L-mandelate, cinnamate or benzoate.)
(xii) Muscarinic antagonists:—Aclidinium bromide, Glycopyrrolate (such as R,R-, R,S-, S,R-, or S,S-glycopyrronium bromide), Oxitropium bromide, Pirenzepine, telenzepine, Tiotropium bromide, 3(R)-1-phenethyl-3-(9H-xanthene-9-carbonyloxy)-1-azoniabicyclo[2.2.2]octane bromide, (3R)-3-[(2S)-2-cyclopentyl-2-hydroxy-2-thien-2-ylacetoxy]-1-(2-phenoxyethyl)-1-azoniabicyclo[2.2.2]actane bromide, a quaternary salt (such as [2-((R)-Cyclohexyl-hydroxy-phenyl-methyl)-oxazol-5-ylmethyl]-dimethyl-(3-phenoxy-propyl)-ammonium salt, [2-(4-Chloro-benzyloxy)-ethyl]-[2-((R)-cyclohexyl-hydroxy-phenyl-methyl)-oxazol-5-ylmethyl]-dimethyl-ammonium salt and (R)-1-[2-(4-Fluoro-phenyl)-ethyl]-3-((S)-2-phenyl-2-piperidin-1-yl-propionyloxy)-1-azonia-bicyclo[2.2.2]octane salt wherein the counter-ion is, for example, chloride, bromide, sulfate, methanesulfonate, benzenesulfonate (besylate), toluenesulfonate (tosylate), napthalenebissulfonate (napadisylate or hemi-napadisylate), phosphate, acetate, citrate, lactate, tartrate, mesylate, maleate, fumarate or succinate)
(xiii) p38 Inhibitors:—681323, 856553, AMG548 (2-[[(2S)-2-amino-3-phenylpropyl]amino]-3-methyl-5-(2-naphthalenyl)-6-(4-pyridinyl)-4(3H)-pyrimidinone), Array-797, AZD6703, Doramapimod, KC-706, PH 797804, R1503, SC-80036, SCIO469, 6-chloro-5-[[(2S,5R)-4-[(4-fluorophenyl)methyl]-2,5-domethyl-1-piperazinyl]carbonyl]-N,N,1-trimethyl-α-oxo-1H-indole-3-acetamide, VX702 and VX745 (5-(2,6-dichlorophenyl)-2-(phenylthio)-6H-pyrimido[1,6-b]pyridazin-6-one);
(xiv) PDE Inhibitors:—256066, Arofylline (3-(4-chlorophenyl)-3,7-dihydro-1-propyl-1H-Purine-2,6-dione), AWD 12-281 (N-(3,5-dichloro-4-pyridinyl)-1-[(4-fluorophenyl)methyl]-5-hydroxy-α-oxo-1H-indole-3-acetamide), BAY19-8004 (Bayer), CDC-801 (Calgene), Celgene compound ((βR)-β-(3,4-dimethoxyphenyl)-1,3-dihydro-1-oxo-2H-isoindole-2-propanamide), Cilomilast (cis-4-cyano-4-[3-(cyclopentyloxy)-4-methoxyphenyl]-cyclohexanecarboxylic acid), 2-(3,5-dichloro-4-pyridinyl)-1-(7-methoxyspiro[1,3-benzodioxole-2,1′-cyclopentan]-4-yl)ethanone (CAS number 185406-34-2)), (2-(3,4-difluorophenoxy)-5-fluoro-N-[cis-4-[(2-hydroxy-5-methylbenzoyl)amino]cyclohexyl]-)-3-pyridinecarboxamide), (2-(3,4-difluorophenoxy)-5-fluoro-N-[cis-4-[[2-hydroxy-5-(hydroxymethyl)benzoyl]amino]cyclohexyl]-3-pyridinecarboxamide,), CT2820, GPD-1116, Ibudilast, IC 485, KF 31334, KW-4490, Lirimilast ([2-(2,4-dichlorobenzoyl)-6-[(methylsulfonyl)oxy]-3-benzofuranyl])-urea), (N-cyclopropyl-1,4-dihydro-4-oxo-1-[3-(3-pyridinylethynyl)phenyl]-)-1,8-naphthyridine-3-carboxamide), (N-(3,5-dichloro-4-pyridinyl)-4-(difluoromethoxy)-8-[(methylsulfonyl)amino])-1-dibenzofurancarboxamide), ONO6126, ORG 20241 (4-(3,4-dimethoxyphenyl)-N-hydroxy-)-2-thiazolecarboximidamide), PD189659/PD168787 (Parke-Davis), Pentoxifylline (3,7-dihydro-3,7-dimethyl-1-(5-oxohexyl)-)-1H-purine-2,6-dione), compound (5-fluoro-N-[4-[(2-hydroxy-4-methyl-benzoyl)amino]cyclohexyl]-2-(thian-4-yloxy)pyridine-3-carboxamide), Piclamilast (3-(cyclopentyloxy)-N-(3,5-dichloro-4-pyridinyl)-4-methoxy-benzamide), PLX-369 (WO 2006026754), Roflumilast (3-(cyclopropylmethoxy)-N-(3,5-dichloro-4-pyridinyl)-4-(difluoromethoxy)benzamide), SCH 351591 (N-(3,5-dichloro-1-oxido-4-pyridinyl)-8-methoxy-2-(trifluoromethyl)-5-quinolinecarboxamide), SelCID(TM) CC-10004 (Calgene), T-440 (Tanabe), Tetomilast (6-[2-(3,4-diethoxyphenyl)-4-thiazolyl]-2-pyridinecarboxylic acid), Tofimilast (9-cyclopentyl-7-ethyl-6,9-dihydro-3-(2-thienyl)-5H-pyrazolo[3,4-c]-1,2,4-triazolo[4,3-a]pyridine), TPI 1100, UCB 101333-3 (N,2-dicyclopropyl-6-(hexahydro-1H-azepin-1-yl)-5-methyl-4-pyrimidinamine), V-11294A (Napp), VM554/VM565 (Vernalis), and Zardaverine (6-[4-(difluoromethoxy)-3-methoxyphenyl]-3(2H)-pyridazinone).
(xv) PDE5 Inhibitors:—Gamma-glutamyl[s-(2-iodobenzyl)cysteinyl]glycine, Tadalafil, Vardenafil, sildenafil, 4-phenyl-methylamino-6-chloro-2-(1-imidazolyl)-quinazoline, 4-phenyl-methylamino-6-chloro-2-(3-pyridyl)-quinazoline, 1,3-dimethyl-6-(2-propoxy-5-methanesulphonylamidophenyl)-1,5-dihydropyrazolo[3,4-d]pyrimidin-4-one and 1-cyclopentyl-3-ethyl-6-(3-ethoxy-4-pyridyl)-pyrazolo[3,4-d]pyrimidin-4-one;
(xvi) PPARγ agonists:—Pioglitazone, Pioglitazone hydrochloride, Rosiglitazone Maleate, Rosiglitazone Maleate ((−)-enantiomer, free base), Rosiglitazone maleate/Metformin hydrochloride and Tesaglitizar;
(xvii) Protease Inhibitors:—Alpha1-antitrypsin proteinase Inhibitor, EPI-HNE4, UT-77, ZD-0892, DPC-333, Sch-709156 and Doxycycline;
(xviii) Statins:—Atorvastatin, Lovastatin, Pravastatin, Rosuvastatin and Simvastatin
(xix) Thromboxane Antagonists: Ramatroban and Seratrodast;
(xx) Vasodilators:—A-306552, Ambrisentan, Avosentan, BMS-248360, BMS-346567, BMS-465149, BMS-509701, Bosentan, BSF-302146 (Ambrisentan), Calcitonin Gene-related Peptide, Daglutril, Darusentan, Fandosentan potassium, Fasudil, Iloprost, KC-12615 (Daglutril), KC-12792 2AB (Daglutril) , Liposomal treprostinil, PS-433540, Sitaxsentan sodium, Sodium Ferulate, TBC-11241 (Sitaxsentan), TBC-3214 (N-(2-acetyl-4,6-dimethylphenyl)-3-[[(4-chloro-3-methyl-5-isoxazolyl)amino]sulfonyl]-2-thiophenecarboxamide), TBC-3711, Trapidil, Treprostinil diethanolamine and Treprostinil sodium;
(xxi) ENACs:—Amiloride, Benzamil, Triamterene, 552-02, PSA14984, PSA25569, PSA23682 and AER002.

The medicament may contain a combination of two or more active ingredients, for example a combination of two or more of the specific active ingredients listed in (i) to (xxi) herein above. In a preferred embodiment the medicament contains an active ingredient selected from mometasone, ipratropium bromide, tiotropium and salts thereof, salemeterol, fluticasone propionate, beclomethasone dipropionate, reproterol, clenbuterol, rofleponide and salts, nedocromil, sodium cromoglycate, flunisolide, budesonide, formoterol fumarate dihydrate, terbutaline, terbutaline sulphate, salbutamol base and sulphate, fenoterol, 3-[2-(4-Hydroxy-2-oxo-3H-1,3-benzothiazol-7-yl)ethylamino]-N-[2-[2-(4-methylphenyl)ethoxy]ethyl]propane-sulphonamide, hydrochloride, indacaterol, aclidinium bromide, N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide); N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. di-D-mandelate); a [2-(4-Chloro-benzyloxy)-ethyl]-[2-((R)-cyclohexyl-hydroxy-phenyl-methyl)-oxazol-5-ylmethyl]-dimethyl-ammonium salt (e.g. hemi-naphthalene-1,5-disulfonate); a (R)-1-[2-(4-Fluoro-phenyl)-ethyl]-3-((S)-2-phenyl-2-piperidin-1-yl-propionyloxy)-1-azonia-bicyclo[2.2.2]octane salt (e.g. bromide or toluenesulfonate); or a combination of any two or more thereof.

Specific combinations of active ingredients which may be incorporated in the medicament include:
(a) formoterol (e.g. as fumarate dihydrate) and budesonide;
(b) formoterol (e.g. as fumarate dihydrate) and fluticasone;
(c) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide) and a [2-(4-Chloro-benzyloxy)-ethyl]-[2-((R)-cyclohexyl-hydroxy-phenyl-methyl)-oxazol-5-ylmethyl]-dimethyl-ammonium salt (e.g. hemi-naphthalene-1,5-disulfonate);
(d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide) and a (R)-1-[2-(4-Fluoro-phenyl)-ethyl]-3-((S)-2-phenyl-2-piperidin-1-yl-propionyloxy)-1-azonia-bicyclo[2.2.2]octane salt (e.g. bromide or toluenesulfonate);
(e) N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. di-D-mandelate) and [2-(4-Chloro-benzyloxy)-ethyl]-[2-((R)-cyclohexyl-hydroxy-phenyl-methyl)-oxazol-5-ylmethyl]-dimethyl-ammonium salt (e.g. hemi-naphthalene-1,5-disulfonate);

N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. di-D-mandelate) and a (R)-1-[2-(4-Fluoro-phenyl)-ethyl]-3-((S)-2-phenyl-2-piperidin-1-yl-propionyloxy)-1-azonia-bicyclo[2.2.2]octane salt (e.g. bromide or toluenesulfonate).

The term medicament as used herein refers generally to the one or more components in a canister dispensed as an aerosol when the canister is actuated in an inhaler. Typically the medicament comprises at least an active ingredient and a propellent. In embodiments of the invention, the medicament may comprise components of the medicament that are introduced into the canister before and/or after the medicament propellent is introduced into the canister, thereby providing a medicament consisting of the medicament components and the medicament propellant.

Preferred embodiments of the present invention will now be described, by way of example only, with reference to the accompanying drawings in which:

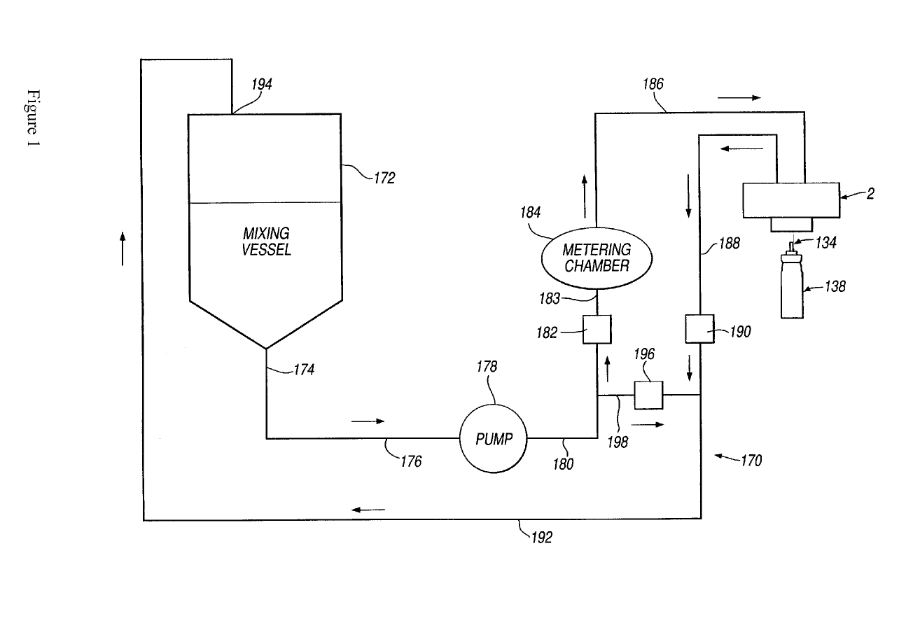

FIG. 1 shows a schematic representation of a filling system, which may be used in accordance with a preferred embodiment of the present invention, for introducing into a container a suspension or solution of a pharmaceutical substance in a propellant under pressure;

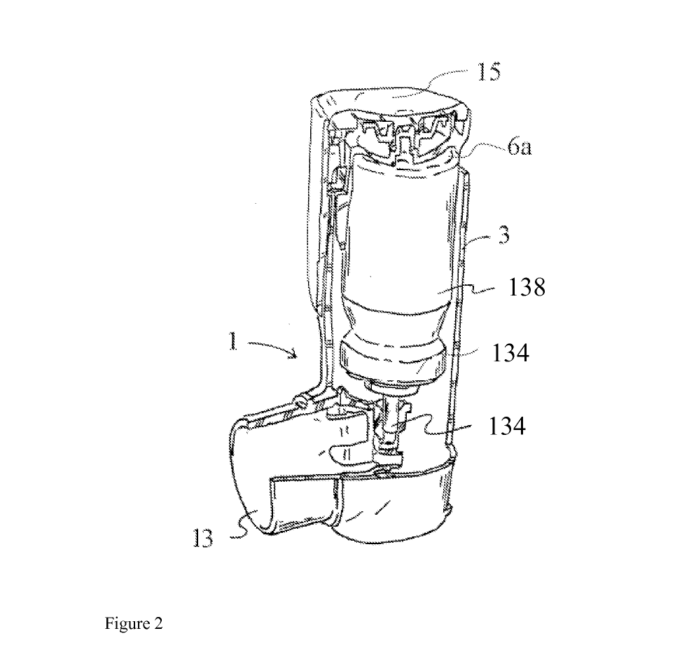

FIG. 2 illustrates schematically a manually-operable pMDI having a canister therein processed in accordance with a preferred embodiment of the present invention;

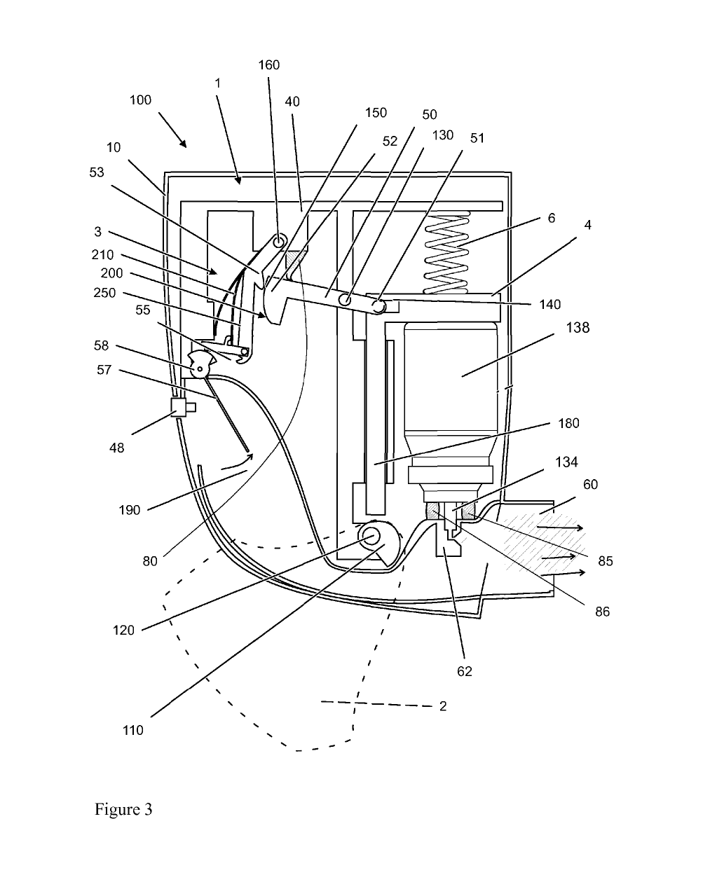

FIG. 3 illustrates schematically an automatically-operable pMDI, which is actuated by a breath-triggered mechanism, having a canister therein processed in accordance with a preferred embodiment of the present invention;

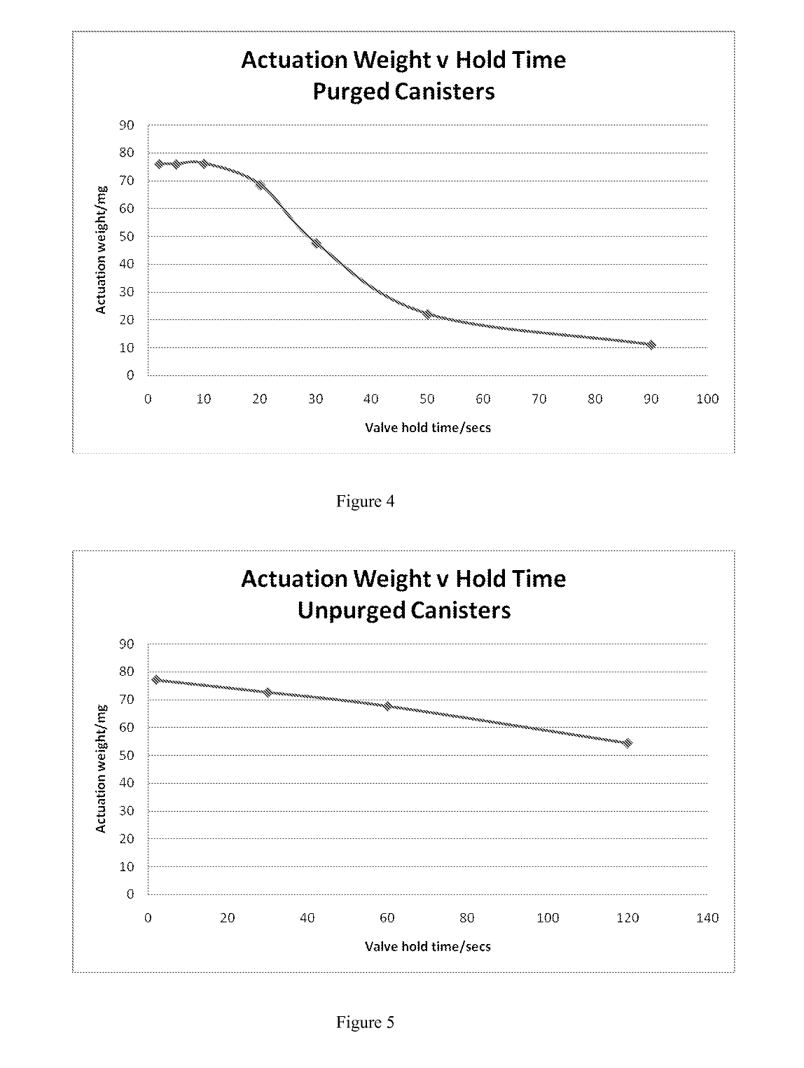

FIG. 4 illustrates actuation weight data obtained for aerosols dispensed from purged canisters after holding the metering valve open for a range of time periods;

FIG. 5 illustrates actuation weight data obtained for aerosols dispensed from an unpurged canister after holding the metering valve open for a range of time periods;

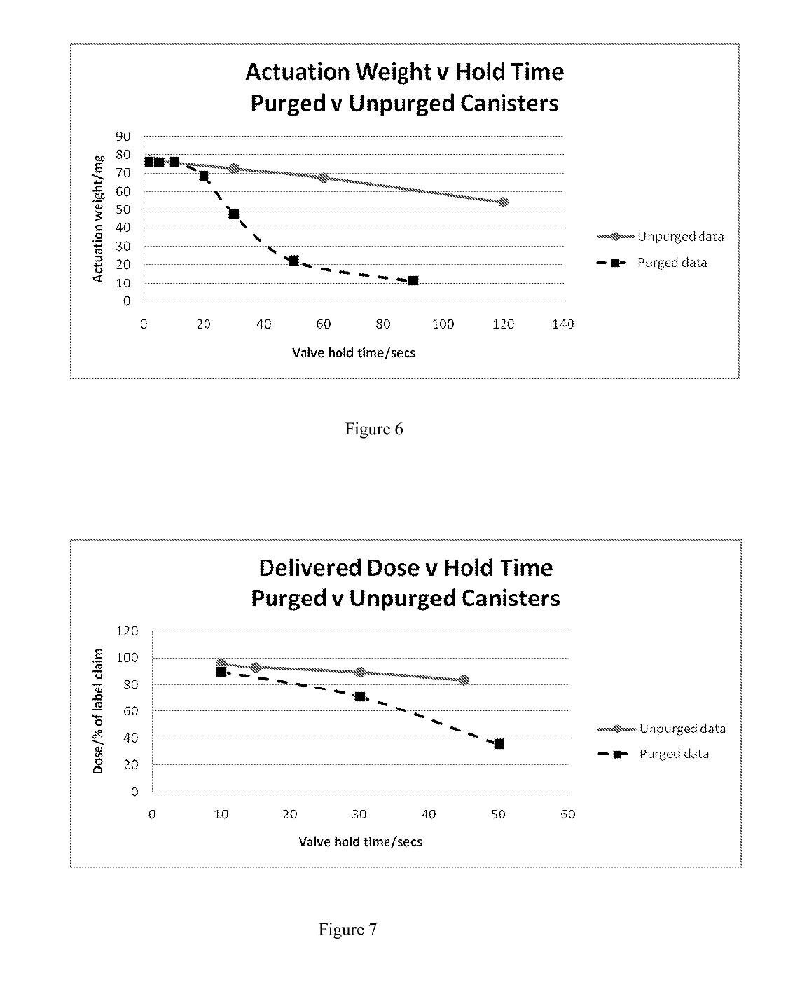

FIG. 6 illustrates the data of FIGS. 4 and 5 on the same axes for ease of comparison; and

FIG. 7 illustrates dose weight data (expressed as a percentage of the dose claimed on the label for that medicament) obtained for medicament dispensed from both purged and unpurged canisters after holding the metering valve open for a range of time periods.

The following describes various, preferred embodiments of the present invention with reference to the figures where appropriate. Like reference numerals are used to indicate like components of preferred embodiments.

FIG. 1 illustrates a known filling system having a filling head 2 for filling a canister 138 with a metered volume of a suspension or solution of a pharmaceutical substance in a propellant under pressure.

The filling head 2 is included in a circulatory line, designated generally by reference sign 170, in which a propellant under pressure containing a pharmaceutical substance in a suspension or solution is circulated. The circulatory line 170 includes a mixing vessel 172 which holds propellant containing pharmaceutical substance in a suspension or solution.

The mixing vessel 172 is pressurised, as is the remainder of the circulatory line 170, so that the propellant is not only under pressure, but is also maintained as a liquid where the boiling point of the propellant is lower than the ambient temperature. A line 176 connects an outlet 174 of the mixing vessel 172 to a pump 178, which pump 178 is provided to pump propellant around the circulatory line 170. Another line 180 connects the pump 178 to the inlet side of an inlet valve 182. A further line 183 connects the outlet side of the inlet valve 182 to a metering chamber 184. The metering chamber 184 is configured to receive a metered volume of the propellant containing pharmaceutical substance in a suspension or solution on opening of the inlet valve 182. The metered volume corresponds to the volume which is required to be introduced into the canister 138 by the filling head 2. A yet further line 186 connects the metering chamber 184 to the filling head 2.

Whilst the embodiment of FIG. 1 illustrates a single-stage pressurised filling process, a two-stage process could be substituted by, for example, providing only propellant under pressure circulating in the lines and having the remaining components of the medicament pre-filled in the canister 138 before sealing the valve to the canister 138 and before filling at filling head 2. Mixing vessel 172 could be omitted for a two-stage process.

Therefore FIG. 1 illustrates a conventional pressure filling process. However, in accordance with the present invention, the canister 138 is not purged of air at any stage prior to reaching the filling head 2. Thus canister 138 is substantially filled with air when the metering valve 134 is sealed to the canister 138 (and the canister may additionally contain one or more components of a medicament, such as a pharmaceutical component (API), co-solvent, surfactant, etc., if the filling process is a two-stage process) and indeed when the canister 138 reaches the filling head 2 and immediately prior to filling. Once a metered amount of propellant (including pharmaceutical component in the one-stage process) is dispensed into the canister 138 by the filling head 2, the filled canister contains a medicament in a suitable dosage formulation as well as air. Typically the contents of a canister in accordance with the present invention will therefore be at higher pressure compared with the contents of a canister in which a conventional purging step is carried to out prior to sealing a canister with a metering valve and will contain significantly more air. Without intending to be bound by theory, it is postulated that this higher pressure might result in better filling of the metering valve and thus, even after the valve has been held in the actuated position for an extended period of time, an improved and more consistent actuation weight of the dispensed aerosol is provided.

Such a canister, as prepared according to the process of FIG. 1, can be used in any suitable, pressurised metered dose inhaler (pMDI). FIG. 2 illustrates schematically a manually-operated inhaler 1 containing a canister 138 having medicament therein for dosing on actuation. The inhaler comprises an actuator body 3 and a mouthpiece 13 through which a user inhales dispensed medicament. This valve rests in a nozzle block at the base of the actuator body 3.

A user actuates the pMDI 1 of FIG. 2 to dispense a dose into the mouthpiece 13 for inhalation by pressing downwardly on the actuator 15 with a finger or thumb, thus depressing the canister 138 which opens the valve 134 and meters a dose out of the nozzle block into the mouthpiece 13 due to the high pressure of the medicament in the canister.

A canister as prepared according to the process of FIG. 1 can also be used in an automatically operated pressurised metered dose inhaler (pMDI). FIG. 3 illustrates schematically a breath-actuated inhaler 1 containing a canister 138 having medicament therein for dosing on actuation automatically in response to breath-triggering of the device.

Briefly, the inhaler 1 comprises a housing 10 containing a breath-triggering mechanism 4, 6, 50-53, 55, 57, 58, 130, 150, 160, 200, 210, 250. The mechanism comprises, inter alia, a breath-triggered flap 57, which rotates about pivot point 58 when a user inhales through the mouthpiece. This enables certain joints 53, 55, 150, 200, 250, to disengage and a link 50 to rotate about its pivot 51. This releases the energy stored in spring 6, which is held in a compressed position until release. The spring 6 forces the engagement 4 to push to downwardly on the canister 138. This compresses the metering valve 134 against the nozzle block 62, thus dispensing a dose of medicament 60 as illustrated.

A more detailed description of the mechanics and the constituents in the illustrated BAI mechanism can be found in WO2008/082359.

### EXAMPLES

To illustrate the advantages of the various aspects of the present invention, both purged and unpurged canisters were tested and the results compared. In order to understand the procedure used it must be appreciated that the phenomenon being studied is partial refilling of the valve metering chamber, not partial emptying. Hence it is the actuation following the one in which the inhaler has been maintained in the actuated position for a period of time that is affected and that must be measured.

### Example 1

Three canisters for three inhalers were assembled and filled with a medicament in the conventional manner, i.e. with a purging step where the canister is purged with propellant, prior to sealing the canister with a metering valve and then filling with the medicament. Each inhaler was tested in the following manner:

1. Actuated in the normal way to dispense an aerosol.

2. Actuated again and maintained in the actuated position (i.e. with the metering valve held open) for a first predetermined time period (recorded in column 1 of Table 1).

3. Weighed.

4. Actuated again and maintained in the actuated position (i.e. with the metering valve held open) for a first predetermined time period (recorded in column 1 of Table 1).

5. Weighed.

6. Actuated in the normal way.

7. Weighed.

The actuation weight, following the hold time, was calculated as the weight of the inhaler at Step 5 minus the weight of the inhaler at Step 3 and the weight of the inhaler at Step 7 minus the weight of the inhaler at Step 5. The mean of the results from the two actuations following each hold time was calculated. Finally the overall mean from the three inhalers was calculated and recorded (in column 2 of Table 1). The process was repeated for each inhaler for the next predetermined time period, and so on to produce the results in Table 1.

TABLE 1

| Time valve held in actuated position | Mean actuation weight (mg) |
|---|---|
| (secs) | following hold time |
| 2 | 76.1 |
| 5 | 76.0 |
| 10 | 76.2 |
| 20 | 68.5 |
| 30 | 47.5 |
| 50 | 22.1 |
| 90 | 11.1 |

As can be seen clearly from the graphical representation (FIG. 4) of the results of Table 1, the actuation weight of a dispensed aerosol subsequent to holding the valve in an actuated state for a time period significantly decreases for longer time periods. This is undesirable because the actuation weight of an aerosol has a direct effect on the delivered dose of the active pharmaceutical ingredient in a medicament and therefore the patient potentially receives a lower drug dose if the metering valve of an inhaler is held open for too long.

### Example 2

For comparison with Example 1, a canister for an inhaler was assembled and filled with propellant in accordance with the aspects of the present invention. The canister was prepared and filled by exactly the same method as for Example 1 except that there was no purging step, so the canister in the inhaler in Example 2 was unpurged. As was the case for Example 1, in Example 2 the inhaler was tested in the following manner:

1. Actuated in the normal way to dispense an aerosol.

2. Actuated again and maintained in the actuated position (i.e. with the metering valve held open) for a first predetermined time period (recorded in column 1 of Table 2).

3. Weighed.

4. Actuated again and maintained in the actuated position (i.e. with the metering valve held open) for a first predetermined time period (recorded in column 1 of Table 2).

5. Weighed.

6. Actuated in the normal way.

7. Weighed.

The actuation weight, following the hold time, was calculated as the weight of the inhaler at Step 5 minus the weight of the inhaler at Step 3 and the weight of the inhaler at Step 7 minus the weight of the inhaler at Step 5. The mean of the results from the two actuations, following each hold time, was calculated and recorded (in column 2 of Table 1). The process was repeated for the next predetermined time period, and so on to produce the results in Table 2. The time periods for Example 2 include longer time periods than for Example 1 but the results are directly comparable.

TABLE 2

| Time valve held in actuated position | Mean actuation weight (mg) |
|---|---|
| (secs) | following hold time |
| 2 | 77.0 |
| 30 | 72.6 |
| 60 | 67.6 |
| 120 | 54.3 |

As can be seen clearly from the graphical representation (FIG. 5) of the results of Table 2, the actuation weight of a dispensed aerosol subsequent to holding the valve in an actuated state for a time period for the Example 2 inhaler does not show such a significant decrease for longer time periods compared with the Example 1 inhalers. Thus the inhaler of Example 2 performed considerably better than the inhalers of Example 1 with respect to more consistent actuation weight of the dispensed aerosols. This can be seen clearly in FIG. 6 which shows the data from the unpurged can (Table 2) versus the data from the purged cans (Table 1).

### Example 3

As discussed above, the actuation weight of an aerosol has a direct effect on the delivered dose of the active pharmaceutical ingredient in a medicament and therefore with the potential drug dose received by the patient per actuation. To verify this, ninety purged canisters and thirty unpurged canisters (for comparison) were prepared in the same manner as above. Each canister held the same predetermined and known number of doses of the chosen medicament, therefore enabling the beginning, the middle and the end of life of the canisters to be determined.

Sixty of the purged canisters were selected for testing. Each canister, which was at the beginning of its life, was placed in an inhaler and actuated normally to prime the metering valve. During the second and third actuation the metering valve was held open for a predetermined period of time of either 15, 30 or 45 seconds (twenty canisters for each time period) and then the inhaler was actuated normally for a fourth time. The delivered dose was measured from the third and fourth actuations combined, using standard inhaler dose collection apparatus at a flow rate of 80 litres per minute. The data was recorded then each canister was actuated a sufficient number of times to bring them each to the middle of their life (i.e. about half the doses were dispensed). The measuring process was then repeated, i.e. each canister was actuated once normally and twice with the metering valve held open for a predetermined period of time of either 15, 30 or 45 seconds. Subsequently the canister in the inhaler was actuated again and the delivered dose was measured from the third and forth actuations combined. The data was recorded then each canister was actuated a sufficient number of times to bring them each to the end of their life (i.e. nearly all of the remaining doses were dispensed). The measuring process was then repeated, i.e. each canister was actuated once normally and twice with the metering valve held open for a predetermined period of time of either 15, 30 or 45 seconds. Subsequently the canister in the inhaler was actuated again and the delivered dose was measured from the third and forth actuations combined. The data from the end of life, the middle of life and the beginning of life was then combined and the mean of these sixty measurements was calculated, as a percentage of the dose claimed on the label for that medication, and is recorded in column 2 of Table 3. For the data relating to the subsequent dose where the metering valve was previously maintained in the actuation position for 10 seconds, thirty unpurged canisters were tested rather than twenty (again at each for the beginning, the middle and the end of life, thus totaling ninety measurements). The mean of this data is also shown in column 2 of Table 3.

For comparison, a similar test was carried out on thirty purged canisters containing the same medicament in the same amount. The tests for the purged canisters were also carried out in the same manner as for the unpurged canisters, in particular they were carried out at the same beginning, middle and end of life stages of the canister lifecycle. The data shown in column 3 of Table 3 is the mean of the thirty data points (three from each of ten canisters) at each hold time. For the purged canisters, the valves were held open for either 10, 30 or 50 seconds prior to the measuring the dose weight from the subsequent actuation. However the data is entirely comparable.

TABLE 3

| | Delivered Dose (% of label claim) | |
|---|---|---|
| Time valve held in actuated | following hold time | |
| position (secs) | Unpurged canisters | Purged canisters |

| | | |
|---|---|---|
| 10 | 95.6 | 89.4 |
| 15 | 93.0 | — |
| 30 | 89.2 | 70.9 |
| 45 | 83.4 | — |
| 50 | — | 35.5 |

As can be seen from FIG. 7, which graphically represents the data of Table 3, the reduction in dose weight of a dose dispensed from an inhaler after the valve is held open in a previous actuation is far less significant when the canister is not purged prior to filling with the medicament. This is advantageous because a patient is less likely to receive a reduced dose of medicament from an inhaler containing an unpurged canister compared with an inhaler containing a purged one, even if the patient accidentally holds of leaves the inhaler with the canister in an actuated state, i.e. with the metering valve open.

1. A process for providing a filled canister for an inhaler, the process comprising the steps of:
providing a canister, the canister comprising an enclosure suitable for containing a medicament and having an opening for receiving the medicament, and the canister being substantially filled with air at ambient conditions;
sealing the opening of the air-filled canister by securing thereto at least one of a metering valve and a sealing mechanism;
providing the sealed, air-filled canister to a filling device; and
dispensing from the filling device a pressurised liquid or gas into the sealed, air-filled canister through at least one of the metering valve and a sealing mechanism, the pressurised liquid and/or gas comprising at least a propellant,
wherein:
the sealed canister is substantially devoid of propellant prior to the step of dispensing the pressurised liquid or gas; and
the sealed canister is substantially filled with at least a first proportion of propellant and a second proportion of air after the step of dispensing the pressurised liquid or gas.

2. The process for providing a filled canister as claimed in claim 1, further comprising the steps of:
providing the air-filled canister to a medicament dispenser, prior to the step of sealing the opening of the air-filled canister; and
dispensing from the medicament dispenser a metered amount of a medicament into the air-filled canister;
wherein the step of sealing the opening of the air-filled canister comprises sealing the opening of the canister containing medicament and air.

3. The process for providing a filled canister as claimed in claim 1, wherein the pressurised liquid and/or gas, dispensed from the filling device into the sealed, air filled canister, further comprises a medicament.

4. A process as claimed in claim 2, wherein the medicament contains an active ingredient selected from mometasone, ipratropium bromide, tiotropium and salts thereof, salemeterol, fluticasone propionate, beclomethasone dipropionate, reproterol, clenbuterol, rofleponide and salts, nedocromil, sodium cromoglycate, flunisolide, budesonide, formoterol fumarate dihydrate, terbutaline, terbutaline sulphate, salbutamol base and sulphate, fenoterol, 3-[2-(4-Hydroxy-2-oxo-3H-1,3-benzothiazol-7-yl)ethylamino]-N-[2-[2-(4-methylphenyl)ethoxy]ethyl]propane-sulphonamide, hydrochloride, indacaterol, aclidinium bromide, N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide); N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (e.g. di-D-mandelate); a [2-(4-Chloro-benzyloxy)-ethyl]-[2-((R)-cyclohexylhydroxy-phenyl-methyl)-oxazol-5-ylmethyl]-dimethyl-ammonium salt (e.g. hemi-naphthalene-1,5-disulfonate); a (R)-1-[2-(4-Fluoro-phenyl)-ethyl]-3-((S)-2-phenyl2-piperidin-1-yl-propionyloxy)-1-azonia-bicyclo[2.2.2]octane salt (e.g. bromide or toluenesulfonate); or a combination of any two or more thereof.

5. The process of claim 2, wherein the medicament comprises at least one excipient.

6. The process of claim 5, wherein the excipient comprises at least one of a surfactant, a co-solvent, and a lubricant.

7. The process of claim 2, wherein the propellant comprises at least one of HFA 227 and HFA 134a.

8. A filled canister for an inhaler, the canister being provided by the process of claim 1.

9. A filled canister as claimed in claim 8, wherein the canister is substantially filled with at least a first proportion of propellant, a second proportion of air and a third proportion of a medicament, the medicament comprising at least one excipient.

10. A filled canister as claimed in claim 9, wherein the excipient comprises at least one of a surfactant, a co-solvent and a lubricant.

11. A pressurised metered dose inhaler, comprising a filled canister as claimed in claim 8.

12. A filled canister as claimed in claim 8, wherein at least an internal surface of the canister is coated.

13. A filled canister as claimed in claim 12, wherein the canister is coated with at least one of a fluoropolymer, perfluoroalkoxyethylene (PFA), polytetrafluoroethylene (PTFE), fluorinated ethylene propylene (FEP), and a PET.

14. A filled canister as claimed in claim 12, wherein the canister is coated by at least one of spraying and by plasma coating.
